(19) United States
(12) Patent Application Publication
(10) Pub. No.: US 2018/0325835 A1
(43) Pub. Date: Nov. 15, 2018

(54) MODIFIED TRANSDERMAL DELIVERY PATCH WITH MULTIPLE ABSORBENT PADS

(71) Applicant: BKR IP Holdco LLC, Broomall, PA (US)

(72) Inventor: Bruce K. REDDING, Jr., Broomalll, PA (US)

(21) Appl. No.: 15/774,224

(22) PCT Filed: Nov. 7, 2016
(86) PCT No.: PCT/US16/60848

§ 371 Date: May 7, 2018

Related U.S. Application Data
(60) Provisional application No. 62/285,668, filed on Nov. 6, 2015.

(51) Int. Cl. A61K 9/70; A61K 9/00; A61K 38/28; A61M 37/00

(52) U.S. Cl. CPC A61K 9/7023; A61K 9/0014; A61M 31/002; A61M 37/0092; A61M 2037/0007; A61K 38/28

(57) ABSTRACT

The invention is modified transdermal patch, which contains two or more absorbent pads placed atop one another within the drag reservoir of the patch, enabling the patch to (1) hold a greater quantity of the drag, (2) extend the useful life of the patch, (3) enhance the quantity of the dose which can be released from the patch, by either passive of active methods of drag release.

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837.

This application hereby incorporates herein by reference the subject matter disclosed in the abstracts, written descriptions, the drawings and claims, in their entireties of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837.

### FIELD OF THE INVENTION

The present invention relates generally to transdermal patches and bandages, also known as drug delivery devices. The present invention relates more specifically to embodiments of modified transdermal patches and bandages which incorporate two or more absorbent pads within the construction of the patch of bandage. The use of absorbent pads within the patch avoid the use of Drug-In-Adhesive patch constructs which place the drug directly into contact with a strong adhesive. Such formulations limit the number of drugs which can be incorporated into a patch as the drug may interact with and become degraded through interaction with adhesive chemicals. Using absorbent pads in a transdermal patch enhance the drug delivery, increase the longevity of the patch and remove the need for adhesive-drug mixtures.

### BACKGROUND OF THE INVENTION

In the prior art, transdermal drug delivery systems employ a medicated device or patch which is affixed to the exposed surface of the skin of a patient. The patch allows a medicinal compound contained within the patch to be absorbed into the skin layers and finally into the patient's blood stream. Transdermal drug delivery avoids the need and the pain associated with drug injections and intravenous drug administration. Transdermal drug delivery also avoids gastrointestinal metabolism of administered drugs, reduces the elimination of drugs by the liver, and provides a sustained release of the administrated drug. Transdermal drug delivery also enhances patient compliance with a drug regimen because of the relative ease of administration and the sustained release of the drug.

Several medicinal compounds are not suitable for transdermal drug delivery since they are absorbed with difficulty through the skin due to the molecular size of the drug or to other bioadhesion properties of the drug. In these cases, when transdermal drug delivery is attempted, the drug may be found pooling merely on the outer surface of the skin and not permeating directly through into the blood stream. Once such example is insulin, which in the prior art has been found difficult to administer by means of transdermal drug delivery.

Some of the most critically needed medications are presently administered either by injection or oral dosage forms. In particular, chemotherapeutic agents are administered in increased dosages because of their need to survive degradation in the gastrointestinal tract. Many critical treatments for AIDS require a cocktail of drugs taken orally in solid dosage forms, several times a day to be effective. These medications are not suitable for transdermal drug delivery use because of the extensive dosing requirement, the inability of the drug molecule to remain stable in a transdermal form. Moreover, the unsuitability for transdermal to skin transfer of the drug leading to low bioabsorbance of the drug across the skin layers.

Generally, conventional transdermal drug delivery methods have been found suitable only for low molecular weight medications such as nitroglycerin for alleviating angina, nicotine for smoking cessation regimens, and estradiol for estrogen replacement in post-menopausal women. Larger molecular medications such as insulin (a polypeptide for the treatment of diabetes), erythropoietin (used to treat severe anemia) and gamma-interferon (used to boost the immune systems cancer fighting ability) are all compounds not normally effective when used with transdermal drug delivery methods of the prior art.

There are three basis designs to transdermal patch products:

### 1. Reservoir Type Patch:

Characterized by the inclusion of a liquid reservoir compartment containing a drug solution or suspension, which is separated from a release liner by a semi permeable membrane and an adhesive.

Commercial examples include: Duralgesic® (Fentanyl), Estraderm @(estradiol) and Transderm-Nitro @ (Nitroglycerin).

2. Matrix Type Patch: Similar to the Reservoir Type Patch design but has two distinguishing characteristics:

1. The drug reservoir is provided within a semisolid formulation.

2. There is no membrane layer.
Commercial Examples include Habitol @ (Nicotine), Nitrodisc @(Nitroglycerine and ProStep @ (Nicotine)

3. Drug-In-Adhesive Type Patch: DIA

Characterized by the inclusion of the drug directly within the skin—contacting adhesive (Wick 1988). In this design the adhesive fulfills the adhesion-to-skin function and serves as the formulation foundation, containing the drug and all the excipients. (Wilking 1994). This category also has two sub-sections: Monolithic and Multilaminate.

Commercial examples include Monolithic DIA: Climara® (Estradiol), Multilaminate DIA: Nicoderm® (Nicotine)

The DIA patch design has several advantages in reducing the size of the overall patch and provides a more concentric seal upon the skin. DIA patches tend to be more comfortable to wear and very thin. A typical DIA patch is 165 to 200 Um thick. Major disadvantages include a longer drug delivery profile. The release of the drug from a DIA patch follows first order kinetics, that is, it is proportional to the concentration of drug within the adhesive. As the drug is delivered from the DIA patch the drug concentration will eventually begin to fall. The delivery rate therefore falls off over time and this fact needs to be considered in the clinical evaluation phase of development.

A major problem with all major forms of transdermal patches is the intermingling of the drug with adhesive compositions. These result in new profiles and in many instances the drug is degraded through the interaction with the adhesive composition. The chemistry of the adhesive can alter the stability, performance and function of certain drugs.

Additionally there are limits to the molecule size of drugs, which can be delivered via a passive system. Typically drug candidates are below 500 Daltons for DIA patches and below 1,000 Daltons for Matrix or Reservoir patches, even through the use of skin enhancers.

### Electronically Assisted Transdermal Devices

There are several approaches used to electronically assist in transdermal delivery including iontophoresis and ultrasound. These systems are designed to either increase the flow of metallic based drugs across the stratum corneum or to microporate the skin or allow the delivery of macromolecules across the stratum corneum into the dermis or underlying tissue. Such electronically assisted transdermal drug delivery devices (TDD's) often use an outside electronic system, which is not connected to a drug-containing patch or the patch has electrodes within it to assist in ionic transfer. Direct connection to a disposable transdermal patch is often impractical because the electrodes or the ultrasonic transducer system is not disposable.

To solve the problem of electronically assisted transdermal drug delivery systems, and enabling such systems to become more portable or wearable by the patient, and in consideration of conventional patch designs wherein drug contamination or denaturing may be caused through interaction with an adhesive or polymer component within the patch design a new transdermal patch, the subject of this invention, was developed.

The use of adhesives, which directly contact the drug, is eliminated in this design. Adhesives may be used in the border of the patch but the DIA, Matrix or Reservoir designs are discarded in favor of an absorbent pad which is held in place in the patch of this invention which also employs a rate control semi-permeable film to provide both on-off functions to the patch and dosing control. The Patch of this invention is also fitted with a snap to enable the patch to connect easily to an ultrasonic emitter. This design enables the more expensive ultrasonic emitter to be retained for future use while the Modified Transdermal Patch is disposable.

Patches are designed to provide either passive or active delivery platforms. The skin has evolved as a formidable barrier against invasion by external microorganisms and against the prevention of water loss. Notwithstanding this, transdermal drug delivery systems have been designed with the aim of providing continuous controlled delivery of drugs via this barrier to the systemic circulation. There are numerous systems now available that effectively deliver drugs across the skin. These include reservoir devices, matrix diffusion-controlled devices, multiple polymer devices, and multilayer matrix systems. This review article focuses on the design characteristics and composition of the main categories of passive transdermal delivery device available.

Mechanisms controlling release of the active drug from these systems as well as patch size and irritation problems will be considered. Recent developments in the field are highlighted including advances in patch design as well as the increasing number of drug molecules now amenable to delivery via this route. From the early complex patch designs, devices have now evolved towards simpler, matrix formulations. One of the newer technologies to emerge is the delivery-optimized thermodynamic (DOT) patch system, which allows greater drug loading to be achieved in a much smaller patch size. With the DOT technology, drug is loaded in an acrylic-based adhesive. The drug/acrylic blend is dispersed through silicone adhesive, creating a semi-solid suspension. This overcomes the problem with conventional drug-in-adhesive matrix patches, in which a large drug load in the adhesive reservoir can compromise the adhesive properties or necessitate a large patch size.

Transdermal drug delivery remains an attractive and evolving field offering many benefits over alternative routes of drug delivery. Future developments in the field should address problems relating to irritancy and sensitization, which currently exclude a number of therapeutic entities from delivery via this route. It is likely that further innovations in matrix composition and formulation will further expand the number of candidate drugs available for transdermal delivery.

Active Transdermal Drug Delivery Market Dynamics
The administration of therapeutic drugs via e advantages of transdermal drug delivery for improving patient compliance, particularly for the treatment of chronic conditions, are well known. But growth of transdermal delivery has been restricted by the need to limit candidate drugs to molecules small enough to effectively pass through the stratum corneum, a limitation that excludes passive transdermal patches as a viable option for the growing number of protein and peptide therapeutic compounds that will represent an increasing share of future NCEs. New technologies that employ energy or mechanical designs to affect drug transport through the skin are expanding the type and number of drug candidates that are viable for transdermal delivery. Evolving active transdermal systems will be well-positioned to address a significant segment of the large-molecule biological drugs expected to emerge from the convergence of automated discovery and genome mapping. As designs shrink in size and become more patient-friendly, opportunities for active transdermal delivery will increase.

Active Transdermal Technology Overview

Market Drivers for Transdermal Delivery

Excipients and Penetration Enhancers

Competitive Landscape

Factors Limiting Growth

Active Transdermal Technologies

Electrical Current

Iontophoresis

Electroporation

Microporation

Lasers

Mechanical Arrays

Radio Frequency

Thermal/Heat

Ultrasound

Active Transdermal Design Factors

Drug Formulation Factors

Proprietary Delivery vs. 3rd Party Patches

Dosing and Rate Factors

Biocompatibility

One objective of the modified transdermal patch of this invention is to avoid Drug-In-Adhesive formulations by utilizing an absorbent pad within the patch. This was disclosed in U.S. Pat. No. 7,440,798, Substance Delivery Device, Bruce K. Redding, Jr. inventor, granted Oct. 21, 2008. However in that work it was discovered that a particular drug, stored within an absorbent material often did not release significant quantities of the drug. While the absorbent pad patch did avoid adhesive-drug combinations, the absorbent pad often released less than 30% of the drug stored within it. This was due to the absorbency power of the absorbent material. Cellulose for example releases up to 50% of the drug, while retaining 50%. Therefore absorbent pad patches were not suitable for long term release patterns for the drug.

### SUMMARY OF THE PRESENT INVENTION

In an effort to increase the longevity of the absorbent pad patch, while also avoiding adhesive-drug combinations, it was discovered that layering absorbent material one layer on top of another or stacking one absorbent pad on top of another, providing a stacked pad design, was very effective in providing long term release and enabling long term patch functionality.

The invention includes embodiments of a modified patch, which contains two or more layers of absorbent material, which in some embodiments are absorbent pads, placed atop one another within the drug reservoir of the patch, enabling the patch to (1) hold a greater quantity of the drug, (2) extend the useful life of the patch, (3) Enhance the quantity of the dose which can be released from the patch, by either passive of active methods of drug release.

This is especially functional with the transdermal delivery of insulin from a patch which is subjected to ultrasonic excitation.

The present invention includes a transdermal delivery device or patch designed with at least two thin layers of an absorbent material or pad, within the drug reservoir compartment, which are stacked on top of one another.

An object of the invention of the invention is a transdermal patch which has a one absorbent pad stacked above the other, or multiple absorbent pads within the patch, which thereby enable greater and longer drug delivery from the patch over time, avoid interaction with adhesive mixes and enhance the quantity of the dose which can be released from the patch, by either passive of active methods of drug release.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 is a diagram illustrating the design of a Reservoir Transdermal Patch.

FIG. 2 is a Drug-In-Adhesive Matrix Construction Patch.

FIG. 3 is an illustration of the structure of human skin.

FIG. 4A is an illustration of a transdermal patch incorporating an absorbent pad construction.

FIG. 4B illustrates weave patterns in absorbent material.

FIG. 4C illustrates an embodiment of a modified substance delivery device incorporating at least two absorbent layers of material within the patch

FIGS. 4D-4M illustrate views of absorbent layers may that may be stacked on top of each other.

FIG. 5A illustrates an exploded view of a transdermal patch incorporating more than one absorbent pad within the patch, stacked, in such a way to increase the holding power of the patch, the delivery rate of the drug from said patch, increase the longevity of the patch and providing for an adhesive free drug interaction.

FIG. 5B is a bottom view of a transdermal patch.

FIG. 5C is a view of a transducer assembly.

FIG. 6 is a Top View depiction of a flexible transdermal patch design modified to use the mesh screen at the bottom of the batch. This particular design uses an absorbent pad to hold the drug and the drug is liberated under an Active control fashion using ultrasound.

FIG. 7 is the Bottom View of a flexible transdermal patch design, incorporating multiple absorbent pads, illustrating the drug reservoir of the patch.

FIG. 8A is an Active transdermal delivery device termed a Patch-Cap, designed to mate with a transducer coupler for the purpose of delivering insulin, employing a double stacked absorbent pad construction, a Screen Mesh fabric at the bottom of the transdermal Patch-Cap.

FIG. 8B illustrates how an ultrasonic transducer coupler is mated to the Patch-Cap illustrated in FIG. 8A, when used with ultrasonically based drug delivery systems.

FIG. 9 is an Active transdermal delivery device which incorporates a two-part design, incorporating a transducer coupler which is slid into or snaps onto a flexible patch, fitted with multiple absorbent pads and a mesh screen for the purpose of delivering insulin.

FIG. 10 is an illustration of a Franz cell, used for collecting a liberated drug from a Transdermal Delivery Device or Patch.

FIG. 11 is an illustration of a single absorbent pad.

FIG. 12 is an illustration of a Dual or multi-absorbent pad system.

FIG. 13 is a flow chart for a method of the invention.

### DESCRIPTION OF THE INVENTION

It is to be understood that the figures and descriptions of the present invention have been simplified to illustrate elements that are relevant for a clear understanding of the present invention, while eliminating, for purposes of clarity, many other elements found in conventional ultrasonic substance delivery systems. Those of ordinary skill in the art will recognize that other elements are desirable and/or required in order to implement the present invention. However, because such elements are well known in the art, and because they do not facilitate a better understanding of the present invention, a discussion of such elements is not provided herein.

As used herein, the term “substance” may include, but are not limited to, any substance, solution or suspension including, but not limited to, a medicinal or non-medicinal substance which may be transported through a live surface or live membrane, including, but not limited to, live tissue and other types of live membranes. The term “delivery device” includes transdermal patches and bandages. The term “proximal” means toward the end of a delivery device where the substance is released from the device. The term “distal” means toward the end of the device that is away from where the substance is released from the device.

Structure of Human Skin and Drug Transport Dynamics.

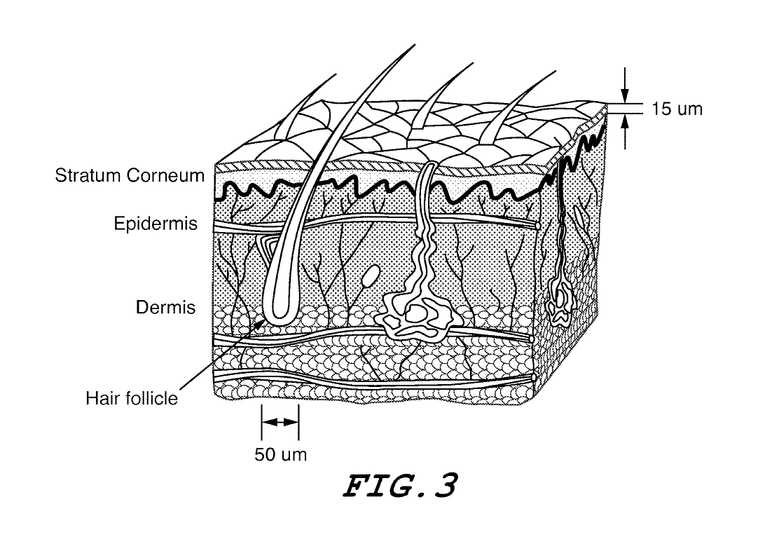

FIG. 3 illustrates the structure of human skin. Essentially there are three pathways through the skin into the bloodstream:
1. Breaching the Stratum Corneum.
2. Passing pharmaceutical agent through pores in the skin.
3. Passing a pharmaceutical agent through the skin by following the hair follicle to the hair root, and from there into the vascular network located at the base of the hair root.

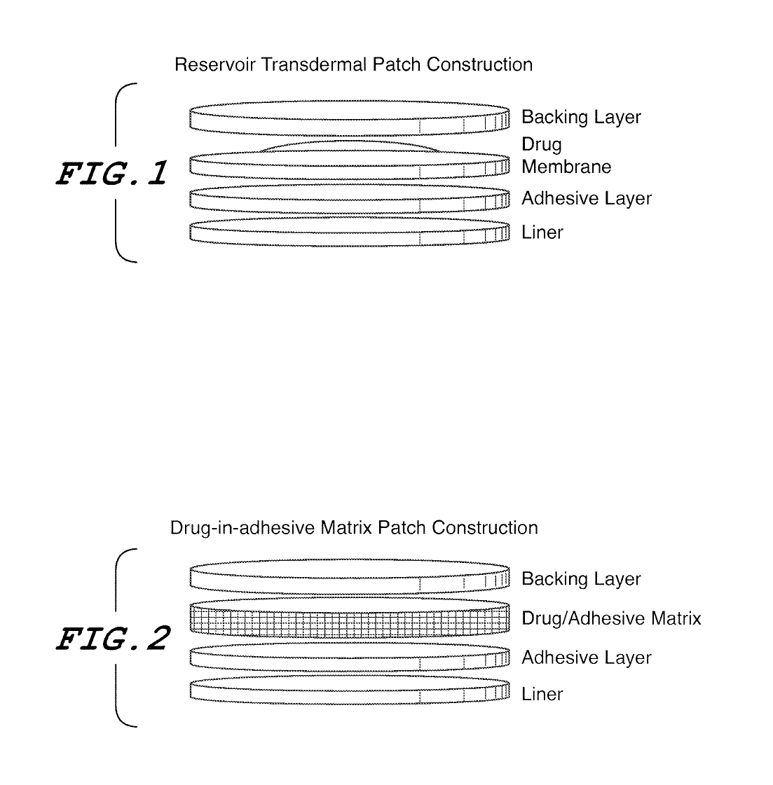

FIG. 1 is a diagram illustrating the design of a Reservoir Transdermal Patch. Characterized by the inclusion of a liquid reservoir compartment containing a drug solution or suspension, which is separated from a release liner by a semi permeable membrane and an adhesive.

Commercial examples include: Duralgesic® (Fentanyl), Estraderm @(estradiol) and Transderm-Nitro® (Nitroglycerin).

A Matrix Type Patch is Similar to the Reservoir Type Patch design but has two distinguishing characteristics:

1. The drug reservoir is provided within a semisolid formulation.

2. There is no membrane layer.
Commercial Examples include Habitol® (Nicotine), Nitrodisc® (Nitroglycerine and ProStep® (Nicotine)

FIG. 2 is a Drug-In-Adhesive Matrix Construction Patch also known as a DIA Patch. Characterized by the inclusion of the drug directly within the skin-contacting adhesive (Wick 1988). In this design the adhesive fulfills the adhesion-to-skin function and serves as the formulation foundation, containing the drug and all the excipients. (Wilking 1994). This category also has two sub-sections: Monolithic and Multilaminate.

Commercial examples include Monolithic DIA: Climara® (Estradiol), Multilaminate DIA: Nicoderm® (Nicotine)

The DIA patch design has several advantages in reducing the size of the overall patch and provides a more concentric seal upon the skin. DIA patches tend to be more comfortable to wear and very thin. A typical DIA patch is 165 to 200 Um thick. Major disadvantages include a longer drug delivery profile. The release of the drug from a DIA patch follows first order kinetics, that is, it is proportional to the concentration of drug within the adhesive. As the drug is delivered from the DIA patch the drug concentration will eventually begin to fall. The delivery rate therefore falls off over time and this fact needs to be considered in the clinical evaluation phase of development.

### Transdermal Delivery Device Incorporating an Absorbent Pad Construction

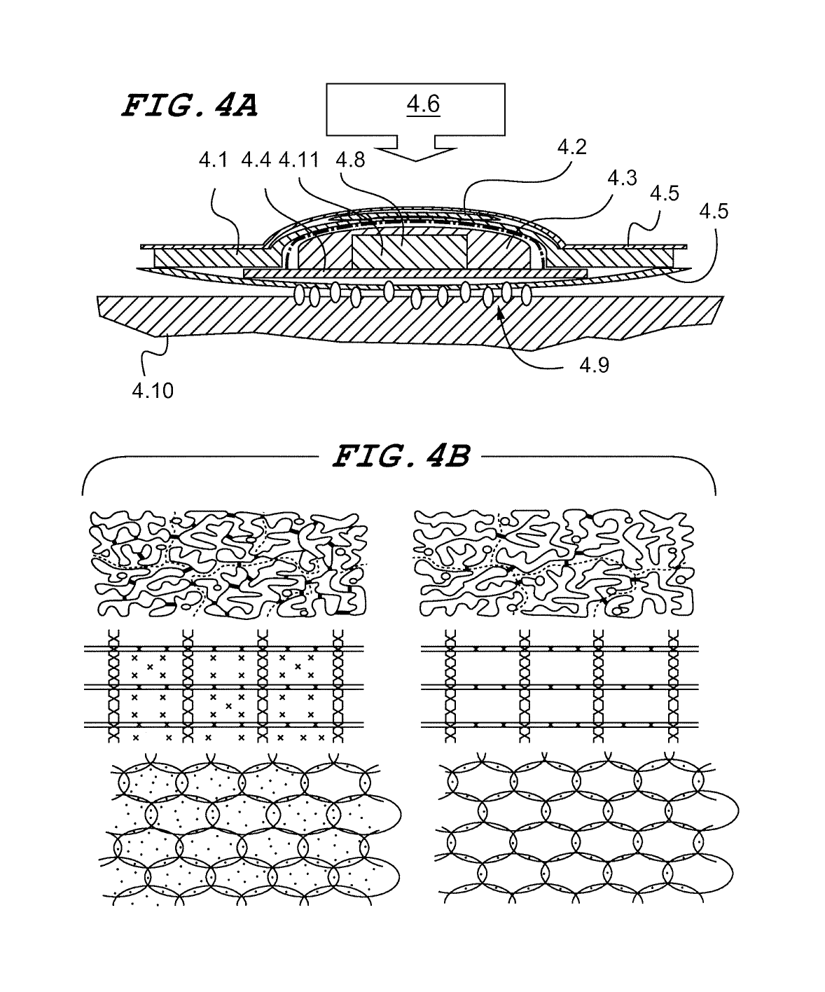

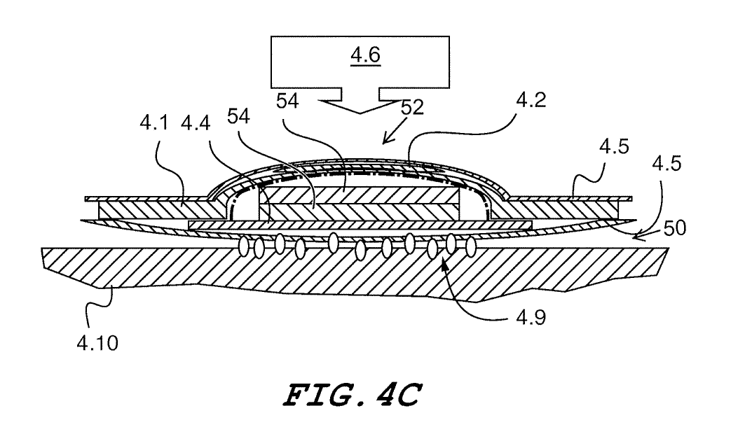

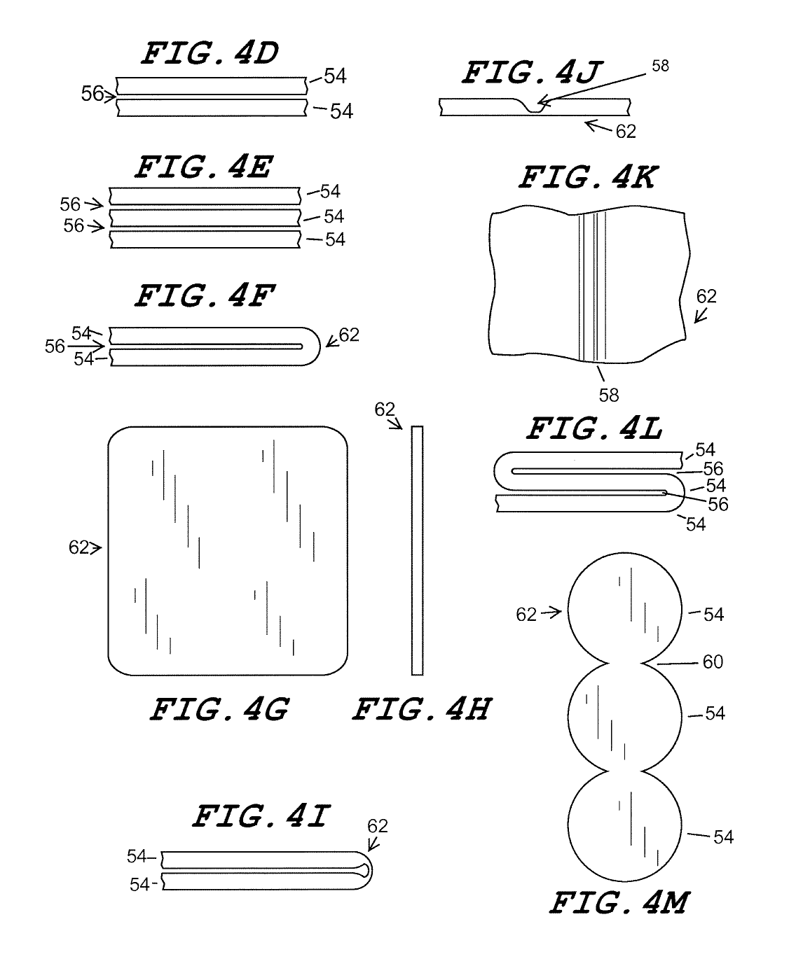

FIG. 4 is a schematic drawing of a transdermal patch incorporating an absorbent pad construction. In some embodiments, the Patch or Transdermal delivery Device (4.1) can include a backbone material (4.1) which holds the materials together, which is comprised itself of a top portion membrane film (4.2), which in this illustration is designed to be used with ultrasound and is termed a sonic membrane (4.2). At the bottom of the patch (4.1) is a Semi-permeable film (4.4) and a Peel Away film (4.5).

Within the patch interior a substance (4.11) is absorbed onto an absorbent pad (4.3), and contained within the drug reservoir compartment (4.8). In an Active Patch embodiment, ultrasound (4.6), or another energy medium, may be delivered through the top membrane (4.2) through the patch (4.1), and will have the effect of liberating the substance (4.9) from the patch (4.) onto the skin's surface (4.10). In this embodiment the substance (4.11) is absorbed and stored within the absorbent pad (4.3) which can hold the drug, free of adhesive interaction for lengthy periods of time depending upon the thickness of the absorbent pad, and its holding power.

Once the substance (4.11) is liberated from the absorbent material (4.3) it travels to the proximal end 50, which may be the bottom, of the patch (4.1) and in some embodiments, through a separator, which can be, but is not limited to, a semi-permeable film layer (4.4) or mesh screen. The proximal end 50 of the patch is the end of the patch toward where the substance is released from the patch. The distal end 52 of the patch is the end of the patch that is generally away from the end were the substance is released from the patch. The semi-permeable film layer (4.4) can be comprised of a film with micro-pores or a mesh screen which will disperse the liberated substance onto droplets (4.9) which then fall onto the skin's surface and are absorbed. In some embodiments, to prepare the patch for use a peel away film 4.5 is peeled away from the bottom of the patch. Once the peel away film (4.5) is removed the flow of the stored drug can be effected in a Passive patch or when an energy media such as ultrasound (4.6) is applied to the Active patch.

Several materials may be used as an absorbent. One preferred material is a cellulose based composition. Common materials such as cellulose, cotton, fabrics, and synthetic fibers such as nylon or sponges may be employed to absorb the medicant, drug or substance. Additionally the weave pattern is a factor in determining the quantity of the drug which can be stored. FIG. 4B is an exemplary illustration of the several weave patterns, known in the art, which may be used in absorbent materials. It is possible to store as much as 100 times the weight of the absorbent material in moisture. Typically a cellulose pad, 1 millimeter thickness and sized 1 inch in diameter will have an absorbency power of 3 X, 3 times its weight in water. Increasing the thickness of the pad, or changing the weave pattern can increase the absorbency power. By choosing the right absorbent material, and adjusting its thickness, a higher absorbency power can be achieved, and therefore a higher dose of the drug can be deposited onto the patch.

A transdermal Patch using the construction illustrated in FIGS. 4A and 4B, and covered by U.S. Pat. No. 7,440,798, Substance Delivery Device, Bruce K. Redding, Jr. inventor, granted Oct. 21, 2008, showed that the delivery of Insulin via ultrasound was very effective using the absorbent pad construct. The pad liberated the insulin in response to an active ultrasonic transmission and when no ultrasound was present the semi-permeable film ceased the delivery of the drug.

However a problem ensued with the patch developed according to FIG. 4B, in that the drug loaded upon the patch tended to deliver up to 50% of the load and not a higher delivery rate. The weave pattern, as exemplarily shown on FIG. 4B, determines the hold capacity for the drug and its eventual release rate under ultrasonic propagation. As the drug was pushed from the absorbent material the residual holding power of the absorbent increased. While this patch avoided the interaction between the drug and the adhesive common in the patch constructs indicated in FIG. 1 and FIG. 2, it was often incapable of long term delivery options as too much of the drug load was retained in the absorbent.

Increasing the thickness of the absorbent pad did not solve this problem. While more drug could be stored with a thicker pad, the result was often that the delivery rate was excessive and once again only 50% of the load could be liberated.

One solution was simply to use multiple thin absorbent at least partially separatable layers, stacked on top of each other. One solution was simply to use multiple thin absorbent pads, stacked on top of each other.

### Delivery Device Including at Least a Double Absorbent Layer

According to embodiments of the present invention, a delivery device is provided for enhancing substance delivery by the use of ultrasound. Use of ultrasonics is particularly effective in delivering larger pharmaceutically active compounds, wherein the delivery device is made to accommodate both the special needs of ultrasonic excitation through the patch construct and the delivery of medicinal compounds stored within the patch.

It is to be understood that in some embodiments the delivery devices can include transdermal delivery of substances and in some embodiments can include the delivery of substances directly on other types of living tissues and membranes. Delivery devices include transdermal patches and bandages.

Reference is now made to FIGS. 4C-4M. FIG. 4C illustrates embodiments of a modified substance delivery device incorporating at least two absorbent layers of material within the patch, wherein the at least two absorbent layers 54 are stacked on top of each other and are at least partially separatable such that a small airspace 56 can be created between at least a portion of the at least two layers. In some embodiments, the at least two layers are stacked in a manner in relationship to each other such that the flow of the absorbed substance is generally perpendicular to the stacked layers or such that the flow of the absorbed substance 4.9 from the absorbent layers to exit the patch requires the substance from the layer or layers distal 52 to the exit area of the device to flow through the layer or layers proximal 50 to the exit area of the device before exiting the patch. In some embodiments, the at least two layers are stacked in a manner in relationship to each other such that the flow of the absorbed substance is generally perpendicular to the stacked layers and such that the flow of the absorbed substance from the absorbent layers to exit the patch requires the substance from the layer or layers distal 52 to the exit area of the device to flow through the layer or layers proximal 50 to the exit are of the device before exiting the device.

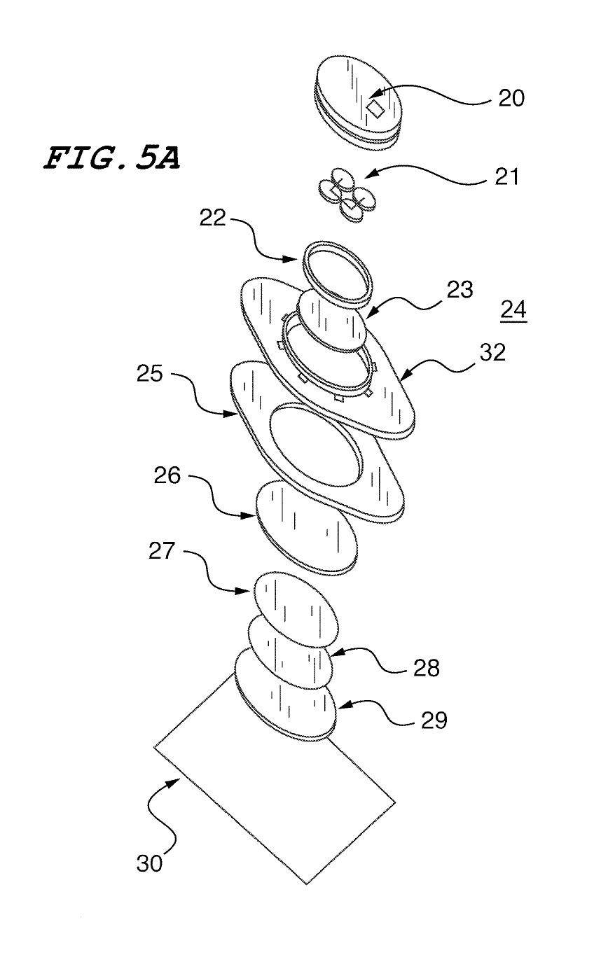

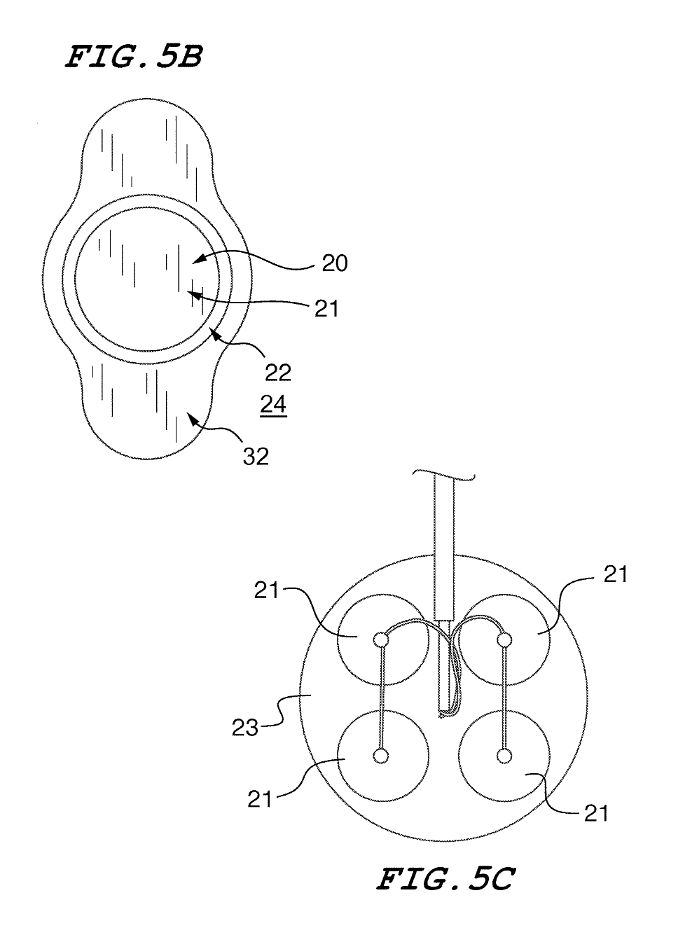

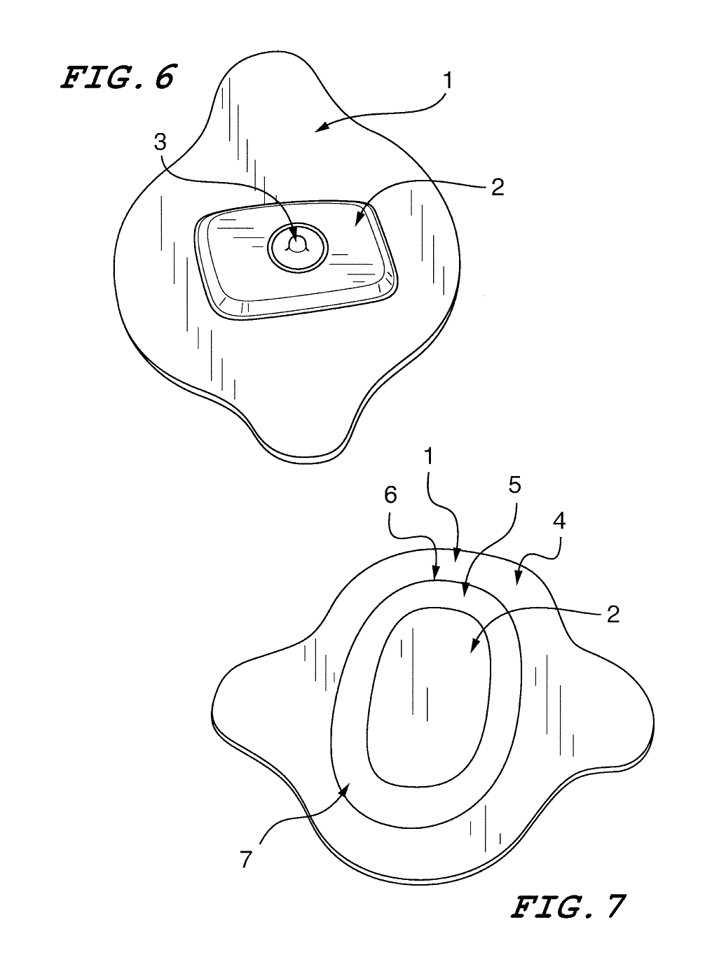

As exemplarily illustrated in FIGS. 4D-4E two or more layers 54 may be stacked on top of each other. It is to be understood that the shape of the absorbent material may be in any variety of shapes. FIG. 5A exemplarily illustrates the absorbent material in the shape of separate circular pads 28. However, it would also be possible to create the stacking with other manners and mechanisms. FIGS. 4F-4M are non-exhaustive exemplary illustrations of other ways that the layers of absorbent material stacking could be accomplished in a transdermal delivery device.

FIGS. 4F-FH and L exemplarily illustrate that at least on piece of absorbent material can be folded over to created the stacked layers. The absorbent material could be folded over once of more than once to create at least two layers, and an increasing number of layers upon additional folding of the material. FIG. 4L illustrates that an absorbent material could be folded two times to create three absorbent layers, and it is to be understood that the invention includes folding an absorbent material additional times to create addition layers, such as but not limited to folding the material three times to create four layers, folding it four times to create five layers, and making additional folds to create additional layers. FIGS. 4I-FJ exemplarily illustrate that that at least one of absorbent material that can be folded over to created the staked layers could have a portion of thinner material 58 at the place where the fold will or does occur, which would remove some of the bulk at the place of the fold.

FIG. 4F-M exemplarily illustrates that a piece of absorbent material could be shaped in a variety of different shapes, including but not limited to a general circular shaped when folded. In addition, there could be a cut-out area 60 between the layers to create the shape and to reduce the material that would be required to be folded.

Reference is now made to FIG. 5A. In this exemplary embodiment, the patch includes a Cap (20) into which an array of transducer discs (21) are placed. A foam ring (22) is affixed to the stainless steel face plate (23) on one end and to the Cap (20) to provide a cushion. The transducer discs (21) are sealed against moisture or air contact within the Cap enclosure 20). The Transducer Cap (20) is thereby removable and can be connected or detached from the transdermal patch (24).

The patch (24) is comprised of a backbone layer of plastic ideally shaped in the form of a butterfly but can be any other shape, An adhesive tape section (25) is cut to the shape of the backbone (24) and comes into contact with the patients skin, but is not in the path of any drug contained with the patch (24).

A membrane film (26) is placed across the backbone layer (32) and acts to protect any stored drug. In the case of an ultrasonic patch the membrane film (26) enables ultrasound to pass through the film without deflection.

Next at least two absorbent pads, (28) are stacked on top of one another.

At the bottom of the patch (24) a mesh screen (29) is placed and this is then covered over with a Rate Control Film (30).

One embodiment is a butterfly patch design, with a snap in ultrasonic transducer when used for a substance delivery system, which in some embodiments could be an insulin delivery system, powered by ultrasound. This is shown in the photograph in FIG. 5B.

FIG. 5C Shows the design of a Transducer Coupler, which is comprised of a stainless steel face plate (23) onto which one or more, four showing, transducer discs are placed. The transducers disc (21) may be a magneto restrictive or piezoelectric transducer crystal, which will convert an electric signal into mechanical forces, sufficient top drive a drug stored on the absorbent pads (28) from the confines of storage within the patch (24), liberated from the patch and onto the surface of the skin of the patient.

While an active patch, powered by ultrasound is shown in FIG. 5A, it should be apparent that this invention can be utilized with other energy transmissions besides ultrasound including radio frequency, light, laser and heat therapy transmission devices.

While an active patch, powered by ultrasound is shown in FIG. 5A, it should be apparent that this invention can be utilized in a passive delivery application as well and is not limited to active delivery systems,

### Transdermal Delivery Device Incorporating an Alternative Flexible Patch Design with Snap-Connector to a Transducer Coupler Capability

FIG. 6 is a Top View depiction of a flexible transdermal patch design modified to use the mesh screen at the bottom of the batch. This particular design uses two absorbent pads to hold the drug and the drug is liberated under an Active control fashion using ultrasound. In this design the flexible patch may be used passively with low molecular weight drugs, generally below 1,000 Daltons. On the top of the patch (6.1) is a snap (6.3) which can attach the patch (6.1) to an ultrasonic transducer device, which in turn sends an ultrasonic transmission through the patch and liberates the drug stored within the absorbent pad section (6.2) onto the surface of the skin.

In FIG. 7, the rear side of the flexible patch is shown. The backbone (7.1) or border of the patch which comes into contact with the skin has an adhesive border (7.4) to stick the patch to the skin's surface. The adhesive (7.4) does not come into contact with the drug directly. The absorbent pad or stacked pads (7.2) is placed within a bordered well or reservoir section (7.5) which isolates it from contact with the adhesive layer (7.4) through the use of a gasket (7.6). At the very bottom of the patch a mesh screen (7.7) is placed across the drug reservoir (7.5) and over the absorbent pad or collection of stacked absorbent pads (7.2). Using this construction a transdermal patch forms minute droplets of the drug upon the skin's surface as depicted in FIG. 4, either through passive or active means. This design patch is especially suited for ultrasonic drug delivery.

### Patch-Cap Design Transdermal Delivery Device

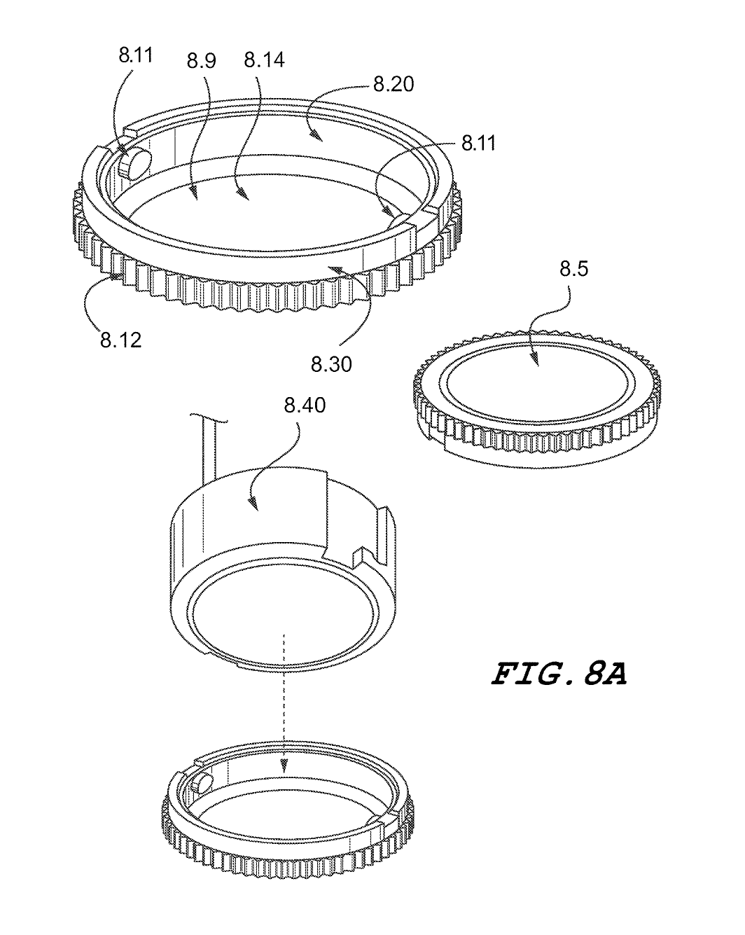

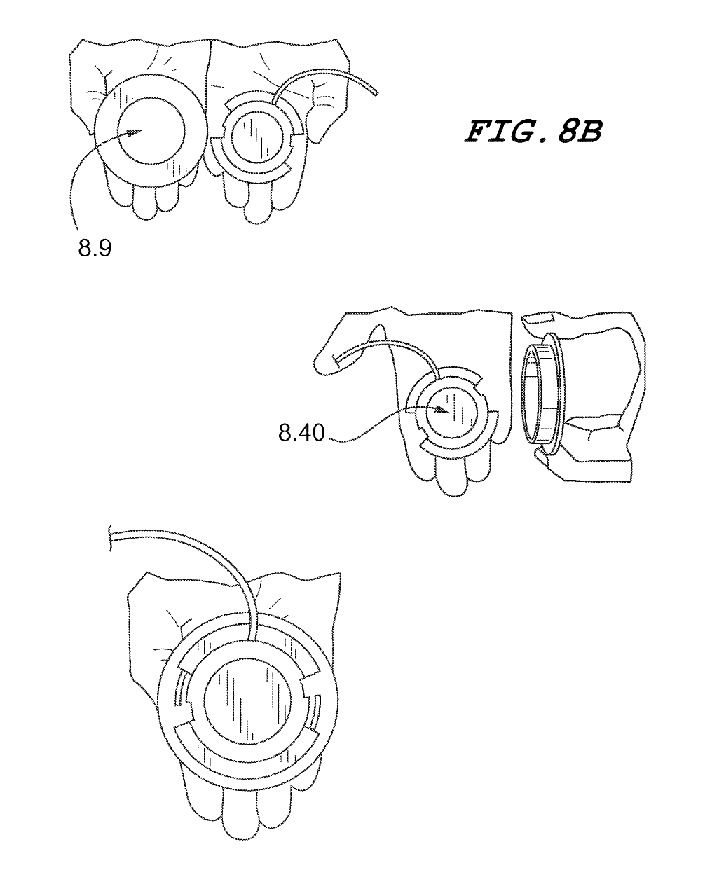

FIG. 8A is an Active transdermal delivery device termed a Patch-Cap, designed to mate with a transducer coupler for the purpose of delivering insulin, employing a Screen Mesh fabric at the bottom of the transdermal Patch-Cap.

FIG. 8B illustrates how the Transducer coupler is mated to the Patch-Cap illustrated in FIG. 8A.

FIG. 8A is an illustration of a Transdermal Patch Cap designed to deliver insulin transdermally using ultrasonic propagation. The Patch Cap includes two absorbent pads (14), which are placed one top of the other (one is shown in the drawing) and which are placed within a holder, the outer snap ring (8.30). It is to be understood that the Transdermal Patch Cap could also be used with one absorbent pad 14. In addition, in some embodiments more than two absorbent pads 14 could be included. In addition, in some embodiments, on absorbent material could be formed into more than one layer of absorbent substance as exemplarily illustrated in FIGS. 4F-M. It is locked into place by an inner snap ring (8.20) and then is used to absorb a substance. This device can be used for insulin delivery. The Cap (8.9) has threaded sides (8.12) in one embodiment and a cap connector grove (8.11) which fits into an ultrasonic emission transducer coupler (8.40). The mesh screen (8.5) is placed across the absorbent pad (8.14) at the bottom of the patch cap. Using this construction a transdermal patch-cap forms minute droplets of the drug upon the skin's surface as depicted in FIG. 4B, either through passive or active means. This design patch is especially suited for ultrasonic drug delivery.

FIG. 8B illustrates how an ultrasonic transducer coupler is mated to the Patch-Cap illustrated in FIG. 8A, when used with ultrasonically based drug delivery systems.

### Active Transdermal Delivery Device which Incorporates a Two-Part Design, Incorporating a Transducer Coupler which is Slid into or Snaps onto a Flexible Patch

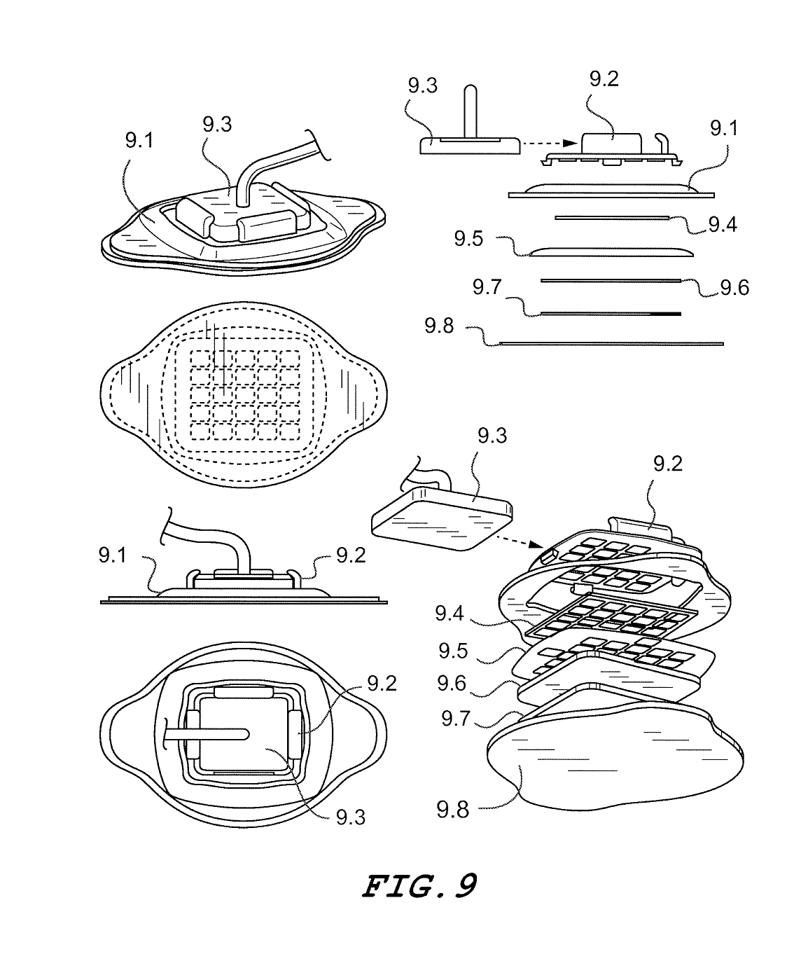

FIG. 9 is an Active transdermal delivery device which incorporates a two-part design, incorporating a transducer coupler which is slid into or snaps onto a flexible patch, fitted with multiple absorbent pads and a mesh screen for the purpose of delivering insulin.

A Backbone layer (9.1) is the base of the patch. A transducer assembly (9.3) snaps onto the patch (9.1) at the top of the patch by connecting to the well cap (9.2) Directly at the top of the patch a film which may allow ultrasound to penetrate it (9.6) is placed directly above the Absorbent Well (9.5), which contains at least two absorbent pads, on top of the other, into which a dose of a particular substance or drug may be stored. In the initial application of this design insulin is stored within each absorbent pad so that the patch may be used to treat diabetes. In addition, in some embodiments more than two absorbent pads 14 could be included. In addition, in some embodiments, on absorbent material could be formed into more than one layer of absorbent substance as exemplarily illustrated in FIGS. 4F-M. A sealing gasket (9.4) can be placed around the well (9.5) to isolate it from any adhesive used in the border of the patch (9.1). A mesh screen (9.7) is installed at the bottom of the patch ahead of a peel away film (9.8) It is to be understood that in some embodiments of this device, the mesh screen and/or the peel away film could be omitted.

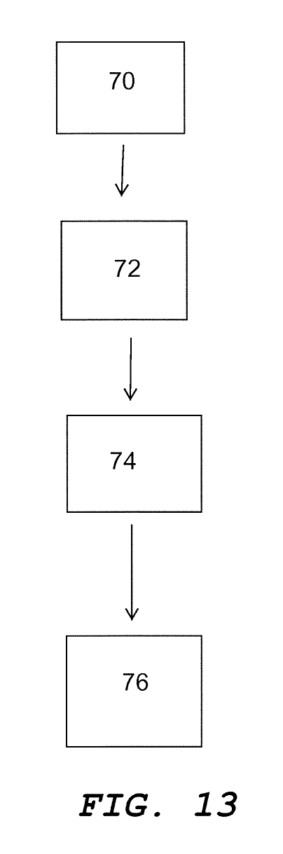

Referring now to FIG. 13, a flow chart for a method of the invention is illustrated. Embodiments of the method of the invention include, but are not limited to, utilization of the devices, dimensions, function, design and materials described in the device embodiments of the invention. In one embodiment, the method includes providing 70 a substance delivery device, providing 72 at least two layers of an absorbent material on to which a substance has been absorbed, placing 74 the device adjacent to a living tissue or next to a material through which the substance my flow to access living tissue, and allowing 76 the release of the substance to access the living tissue.

### EXPERIMENTS

### Comparison of Patch-Capsutilizing a Single Absorbent Pad Vs. A Double Absorbent Pad Construction, Delivery of Insulin when Propagated by Ultrasound, Using the Patch-Cap Construction Indicated In FIGS. 8A and B

### Experiment 1

### Experiment Number: BKR-1011-001

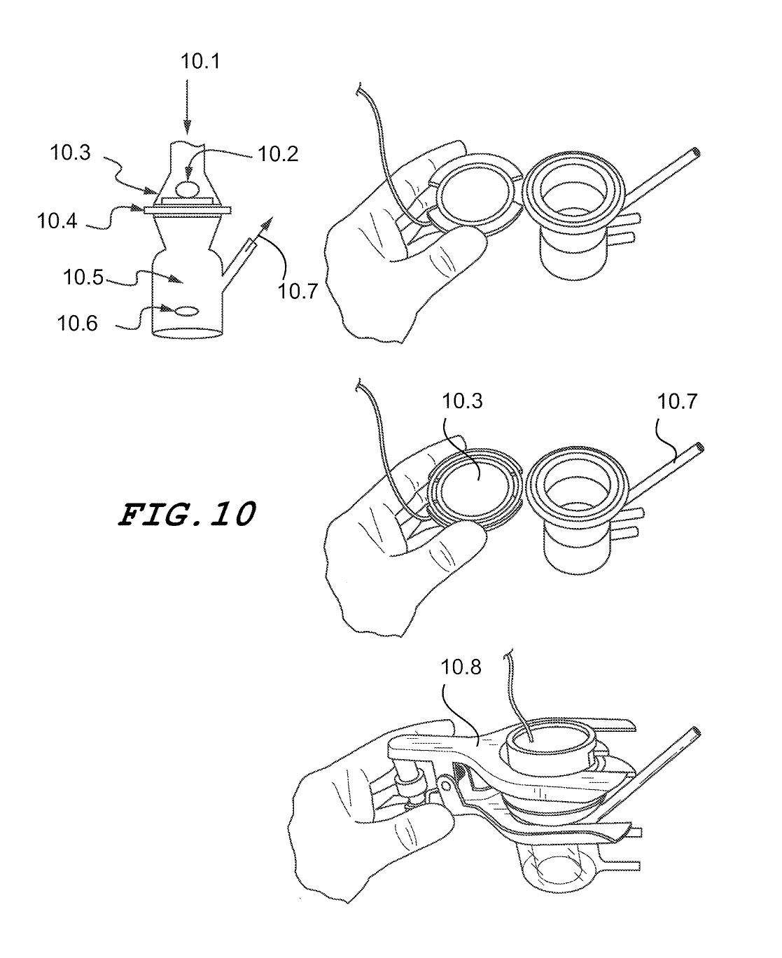

FIG. 10 is an illustration of a Franz cell, used for collecting a liberated drug from a Transdermal Delivery Device or Patch. In FIG. 10 Ultrasound (10.1) is delivered from a transducer (10.2) to a patch (10.3) mounted on a flask (10.5). A sample of human skin (10.4) can be placed under the patch (10.3). Ultrasound traveling through the patch will liberate drugs stored within the patch, which will then flow through the skin section and into the receiver compartment of the flask (10.5). There a magnetic stir bar (10.6) is used to circulate the liberated drug in the receiver compartment (10.5). Samples of the liberated drug may be taken from the receiver compartment (10.5) via the sampling port (10.7). A clamp (10.8) is used to hold the Patch onto the Franz cell.

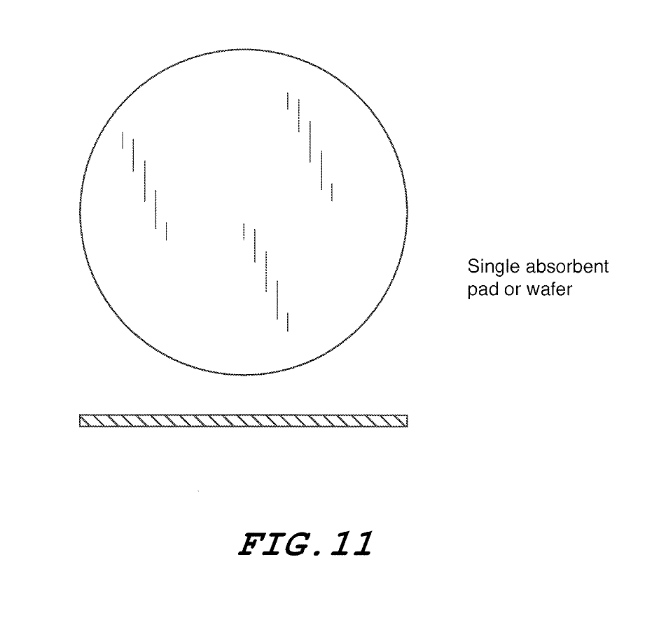

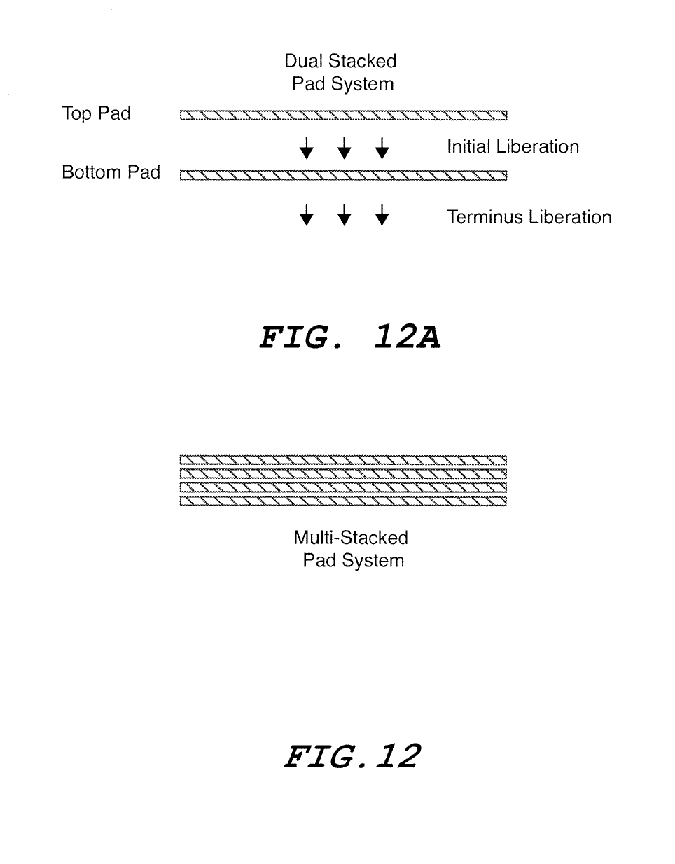

In this experiment an absorbent system was compared, one using a single absorbent pad and one using a stacked absorbent pad. FIG. 11 is an illustration of a single absorbent pad system. FIG. 12 is an illustration of a Dual or multi-absorbent pad system. In this experiment an absorbent pad was fabricated according to the following dimensions:

### Patch Cap Specifications:

Composition:

Polyvinylidene Chloride Saran ™ Film
Absorbent Material
Protective Backing (To be specified)

Sandwiched pad comprising of a top section comprised of Saran™ Polyvinylidene Chloride film, followed by a mid-section absorbent material comprised of 100% virgin wood pulp cellulose fiber supplied by Buckeye Products Co, and a synthetic emulsion binder and an under section which includes a of protective film.

### Sonic Membrane Specifications:

Manufacturer: SC Johnson
Brand Name: Saran™ Classic
Chemical Name: Polyvinylidene Chloride
Thickness: 0.5 mil

### Absorbent Material Physical Properties: BKR-1011-3

Pad Material: Woven cellulose fiber, un-bleached
Pad Diameter: 2.25 inch

### Pad Thickness: 0.92 mm Thickness

| Basis Weight | 70 | g/m2 |
|---|---|---|
| MQ3RD001 | | |
| Estimated Slit Thickness | 0.92 | mm/ply |
| MQ3RD002 | | |
| MD Dry Tensile | 750 g/25.4 mm | |
| MQ3RD003 | | |
| CD Dry Tensile | 635 g/25.4 mm | |
| MQ2RD003 | | |
| CD Wet Tensile | 310 g/25.4 mm | |
| MQ3RD004 | | |
| Absorbency Rate | 3.0 | seconds (water) |
| MQ3RD005 | | |
| Absorbency Capacity | 12.0 | g/g (water) |
| MQ3RD005 | | |

| | |
|---|---|
| Brightness | 81% |
| MQ3RD007 | |

Holding Capacity: 100 units insulin
(Humalog R supplied by the Lilly Company)
(100 units/cc)

In Experiment 1 a Patch-Cap is configures according to FIG. 8A using a single absorbent wafer as shown in FIG. 11. 100 units of Humalog insulin is loaded onto the absorbent pad and the Patch cap configured onto a test Franz cell, as shown in FIG. 10, with no human skin utilized in this experiment. The transducer array was a 4 transducer coupler as shown in FIG. 5C, being model Number Piezoelectric Transducer Standard Array for U-Strip UTDD, Model No: EX1-4 manufactured by Transdermal Specialties Co., and powered by an ultrasonic driver device manufactured by Transdermal Specialties Co. and known as U-STRIP DESKTOP ULTRASONIC DRIVER, Model No. ESI-25.

The Patch-Cap, single absorbent pad is loaded with 100 units of Lispro insulin (Humalog R-100 supplied by Eli Lilly Co.) and is powered by an ultrasonic applicator for a total of 8 hours.

### Ultrasonic Settings & Single Absorbent Pad

| | |
|---|---|
| Ultrasonic Frequency: | 23 kHz |
| Intensity: | 500 mW/sq. cm |
| Ultrasonic Transmission | Alternating between 50 milliseconds |
| | saw tooth and 50 milliseconds square |
| | wave form. This alternation avoid |
| | cavitation or over heating of the insulin |
| | within the Patch-Cap. |
| Dimensions of Patch-Cap | 2.25 inch diameter absorbent pad area |
| Absorbent pad used | BKR-1011-003 Cellulose, 1 mil |
| | thickness |
| Number of absorbent | One |
| pads in Patch-Cap | |
| Duration of Experiment: | 8 hours under continuous ultrasound |
| Filter Screen used | 100 × 100 mesh on bottom of Patch- |
| | cap |

The delivery pattern of the drug upon the surface of the skin corresponded to the pooling effect shown in FIG. 4B.

### Results:

| | |
|---|---|
| Duration of Ultrasonic exposure | 8 hours total |
| First 4 hours Delivery Rate (Insulin | 7.2 units of insulin per hour |
| collected from Franz Cell) | for a total of 28.8 units |
| | collected at hour 4 |
| Second 4 hour Period Delivery Rate | 4.3 units of insulin per hour |
| (Insulin collected from Franz Cell) | for a Total of 17.2 units |
| | collected between Hour 4 |
| | and at hour 8 |
| Total cumulative amount of insulin | 46 units |
| collected after 8 hours | |
| Balance of insulin remaining in | 54 units |
| absorbent pad system(single pad) | |
| Third 4 hour period Delivery Rate | 1.2 units of insulin per hour |
| (Insulin collected from Franz Cell) | for a total of 4.8 units |
| | collected between hour 8 |
| | and at hour 12 |
| Total accumulated amount of insulin | 50.8 units |
| collected after 12 hours | |
| Balance of insulin remaining in | 49.2 units. |
| absorbent pad system(single pad) | |

Note:
no quantity of insulin could be liberated after hour 12.

Using a single absorbent pad construction, after 8 hours of ultrasonic driving power, the patch-Cap could release only 50.8% of the load of insulin upon the absorbent pad.

For a 200 lb. man, with a maximum need of 3.8 units of Lispro insulin per hour the single absorbent patch-cap could only be relied to work for 12 hours or to deliver a maximum of 46 units before the delivery rate from the patch would fall below the listed need for the average 200 lb. male diabetic.

### Experiment 2

### Experiment Number: BKR-1011-002

In Experiment 2 a Patch-Cap is configures according to FIG. 8A using a single absorbent pad, at 2 mm thickness. 100 units of Humalog insulin is loaded onto the absorbent pad and the Patch cap configured onto a test Franz cell, as shown in FIG. 10, with no human skin utilized in this experiment. The transducer array was a 4 transducer coupler as shown in FIG. 5C, being model Number Piezoelectric Transducer Standard Array for U-Strip UTDD, Model No: EX1-4 manufactured by Transdermal Specialties Co., and powered by an ultrasonic driver device manufactured by Transdermal Specialties Co. and known as U-STRIP DESKTOP ULTRASONIC DRIVER, Model No. ESI-25.

The Patch-Cap, a thicker, single absorbent pad, at 2 mm thickness, is loaded with 100 units of Lispro insulin (Humalog R-100 supplied by Eli Lilly Co.) and is powered by an ultrasonic applicator for a total of 8 hours.

### Ultrasonic Settings & Double Absorbent Pad

| | |
|---|---|
| Ultrasonic Frequency: | 23 kHz |
| Intensity: | 500 mW/sq. cm |
| Ultrasonic Transmission | Alternating between 50 milliseconds |
| | saw tooth and 50 milliseconds square |
| | wave form. This alternation avoid |
| | cavitation or over heating of the insulin |
| | within the Patch-Cap. |
| Dimensions of Patch-Cap | 2.25 inch diameter absorbent pad area |
| Absorbent pad used | A single BKR-1011-003 Cellulose, 2.0 |
| | mil thickness |
| Number of absorbent | One |
| pads in Patch-Cap | |
| Duration of Experiment: | 8 hours under continuous ultrasound |
| Filter Screen used | 100 × 100 mesh on bottom of Patch- |
| | cap |

The delivery pattern of the drug upon the surface of the skin corresponded to the pooling effect shown in FIG. 4B.

### Results:

| | |
|---|---|
| Duration of Ultrasonic exposure | 8 hours total |
| First 4 hours Delivery Rate (Insulin | 7.2 units of insulin per hour |
| collected from Franz Cell) | for a total of 28.8 units |
| | collected at hour 4 |
| Second 4 hour Period Delivery Rate | 5.8 units of insulin per hour |
| (Insulin collected from Franz Cell) | for a Total of 23.2 units |
| | collected between Hour 4 |
| | and at hour 8 |
| Total cumulative amount of insulin | 52.0 units |
| collected after 8 hours | |
| Balance of insulin remaining in | 48 units |
| absorbent pad system(single pad) | |
| Third 4 hour period Delivery Rate | 4.6 units of insulin per hour |
| (Insulin collected from Franz Cell) | for a total of 18.4 units |
| | collected between hour 8 |
| | and at hour 12 |
| Total accumulated amount of insulin | 70.4 units |
| collected after 12 hours | |
| Balance of insulin remaining in | 29.6 units. |
| absorbent pad system(single pad) | |

Note:
no quantity of insulin could be liberated after hour 12.

### Experiment 3

### Experiment Number: BKR-1011-002

In Experiment 2 a Patch-Cap is configures according to FIG. 8A using a double absorbent wafer as shown in FIG. 12A. 100 units of Humalog insulin is loaded onto the absorbent pad and the Patch cap configured onto a test Franz cell, as shown in FIG. 10, with no human skin utilized in this experiment. The transducer array was a 4 transducer coupler as shown in FIG. 5C, being model Number Piezoelectric Transducer Standard Array for U-Strip UTDD, Model No: EX1-4 manufactured by Transdermal Specialties Co., and powered by an ultrasonic driver device manufactured by Transdermal Specialties Co. and known as U-STRIP DESKTOP ULTRASONIC DRIVER, Model No. ESI-25.

The Patch-Cap, single absorbent pad is loaded with 100 units of Lispro insulin (Humalog R-100 supplied by Eli Lilly Co.) and is powered by an ultrasonic applicator for a total of 8 hours.

### Ultrasonic Settings & Double Absorbent Pad

| | |
|---|---|
| Ultrasonic Frequency: | 23 kHz |
| Intensity: | 500 mW/sq. cm |
| Ultrasonic Transmission | Alternating between 50 milliseconds |
| | saw tooth and 50 milliseconds square |
| | wave form. This alternation avoid |
| | cavitation or over heating of the insulin |
| | within the Patch-Cap. |
| Dimensions of Patch-Cap | 2.25 inch diameter absorbent pad area |
| Absorbent pad used | BKR-1011-003 Cellulose, 1 mil |
| | thickness × 2, STACKED ON TOP OF |
| | ONE ANOTHER |
| Number of absorbent | Two |
| pads in Patch-Cap | |
| Duration of Experiment: | 8 hours under continuous ultrasound |
| Filter Screen used | 100 × 100 mesh on bottom of Patch- |
| | cap |

The delivery pattern of the drug upon the surface of the skin corresponded to the pooling effect shown in FIG. 4B.

### Results:

| | |
|---|---|
| Duration of Ultrasonic exposure | 12 hours total |
| First 4 hours Delivery Rate (Insulin | 16.4 units of insulin per hour |
| collected from Franz Cell) | for a total of 65.6 units |
| | collected at hour 4 |
| Second 4 hour Period Delivery Rate | 15.8 units of insulin per hour |
| (Insulin collected from Franz Cell) | for a Total of 63.2 units |
| | collected between Hour 4 |
| | and at hour 8 |
| Total cumulative amount of insulin | 128.8 units |
| collected after 8 hours | |
| Balance of insulin remaining in | 71.2 units |
| absorbent pad system(single pad) | |
| Third 4 hour period Delivery Rate | 14.6 units of insulin per hour |
| (Insulin collected from Franz Cell) | for a total of 58.4units |
| | collected between hour 8 |
| | and at hour 12 |
| Total accumulated amount of insulin | 187.2 units |
| collected after 12 hours | |
| Balance of insulin remaining in | 12.8 units. |
| absorbent pad system(single pad) | |

Note:
no quantity of insulin could be liberated after hour 12.

### COMPARISON and OBSERVATIONS

Using a double absorbent pad construction, under continuous ultrasonic driving power, the Patch-Cap could release over twice the load of insulin to a diabetic patient.

For a 200 lbs. man, with a maximum need of 3.8 units of Lispro insulin per hour the single absorbent patch-cap could only be relied to work for 12 hours or to deliver a maximum of 46 units before the delivery rate from the patch would fall below the listed need for the average 200 lb. male diabetic.

The use of a thicker pad was not as effective as a double pad use, stacked on top of one another.

For a 200 lbs. man, with a maximum need of 3.8 units of Lispro insulin per hour the double absorbent patch-cap could be relied to work for 49 hours or to deliver a maximum of 187 units before the delivery rate from the patch would fall below the listed need for the average 200 lb. male diabetic.

The use of a double absorbent pad extended the useful delivery capability of the ultrasonically powered insulin patch by a factor of 4.

In fact, coupled with an ultrasonic propagation mechanism this experiment demonstrated significant advantages to the treatment of diabetes, through the use of a transdermal delivery device which is constructed with multiple absorbent pads instead of using one pad or even a pad with a greater overall thickness.

Combination Ultrasonic Device and Transdermal Patch:

The invention further includes a method for conducting the transport of active substances, including but not limited to pharmaceutical compositions, through the body surface of an individual. The method includes applying ultrasound through a transdermal delivery device which is attached with to a programmable ultrasonic regulator device, which itself is worn by the individual wherein said ultrasound is applied at a frequency and intensity and for a time period effective to enable movement of a therapeutic quantity of the active pharmaceutical composition from a transdermal delivery device, or transdermal patch, through the skin, for the purpose of effecting regulated, and timed drug delivery to the individual.

The method of can also include providing an ultrasound having a frequency in the range of about 20 kHz to 10 MHz, and having intensity in the range of about 0.01 W/cm·sup.2 to 5.0 W/cm·sup.2, and wherein the ultrasound is applied either in a continuous or pulsed manner.

The method can further include affixing or connecting the wearable, portable sonic device with a transdermal patch which provides the transdermal delivery of drugs or other substances to the individual. The connection can be effected via the use of a snap-on feature built into the transdermal patch, or by some other effective connector which provides a connection of the backbone of the patch with a transducer or array of transducers.

The method can further include providing that the wearable, portable sonic device is controllable through programmable settings for at least one of the following: the quantity of drug released by the device, the time interval of active ultrasonic drug delivery, the time interval between ultrasonic drug delivery, the frequency and intensity of the ultrasonic signal, the basal delivery schedule of drug dosing and the bolus delivery schedule of booster doses of a particular drug, with both automatic functions and a manual operation capability.

The invention further includes a delivery device for conducting the transport of active substances, including but not limited to pharmaceutical compositions, through the body surface of an individual, which is attachable with a programmable ultrasonic regulator device. The programmable ultrasonic regulator device is wearable by the individual wherein ultrasound is applied through the device at a frequency and intensity and for a time period effective to enable movement of a therapeutic quantity of the active pharmaceutical composition from a transdermal delivery device, or transdermal patch, through the skin or live tissue for the purpose of effecting regulated, and timed drug delivery to the individual. The delivery device can also contain a transducer assembly, holding a single or multiple transducers of any effective type including cymbal type, wherein the transducer assembly may be internal or external to the device.

The invention further includes an ultrasonic drug deliverer that uses a single transducer or an array of transducers, employed to deliver ultrasonic energy through a transdermal patch, wherein the array makes possible the application of the ultrasonic drug transport through a number of multiple skin transport sites. The drug deliverer avoids premature damage to the skin transport sites and effects the greatest quantity of deliverable drug from the patch, through the patients skin and into the bloodstream. In some embodiments, the multiple transducer elements in the drug deliverer transmit ultrasound at identical frequencies and intensity levels to each other. In some embodiments, the multiple transducer elements in the drug deliverer transmit ultrasound at differing frequencies and intensity levels to each other.

The invention includes an ultrasonic substance delivery transdermal patch, wherein the modified transdermal patch includes:

A) Patch Backbone and Sonic membrane:
A backbone of the patch has a section including a membrane which will enable the effective transmission of the ultrasonic signal throughout the patch, said membrane possessing properties which will not interfere with the frequency or reduce the intensity of the ultrasonic transmission, wherein the membrane may be made of a material including saran, or such other polymeric compound which will similarly not interfere with the frequency and intensity of an ultrasonic transmission.

B) Absorbent Pad:
An absorbent compound as a means for storing a substance, including but not limited to a medication, drug or nutrient compound within the patch, wherein an ultrasonic transmission through the patch acts to liberate the substance from the absorbent pad to be transported to the patient through skin permeation.

C) Semi-Permeable Film
A semi-permeable film at the bottom of the patch, at the interface where the patch comes into contact with the patients skin. The semi-permeable film provides a means for delivering a stored substance including but not limited to a medication, drug or nutrient compound from within the patch to the patients skin surface only upon the active generation of ultrasonic transmissions through the patch thereby providing an On-Off function with the propagation of ultrasound through the patch, and a means of regulating the quantity of the substance or dose to the patient, i.e., the control of the delivered dose to the patient, wherein said semi-permeable film is comprised of a material which provides osmotic by-pass, via ultrasonic propagation, or is comprised of a membrane or film possessing perforations which expand in the presence of ultrasound and which contract when ultrasound is terminated, to enable substance delivery.

D) Gasket for providing a good seal to the skin
A gasket around the backbone of the patch, as means of preventing air from reaching under the patch and interfering with the intensity of the ultrasonic transmission through the patient's skin and for preventing leakage of the drug contained within the patch.

In some embodiments of the transdermal patch, the semi-permeable film may be comprised of materials including but not limited to the following materials:

Membranes:
CTA (Cellulose Tri-Acetate)
TFC (Thin Film Composite) sometimes labeled as TFM (Thin Film Membrane).

Reverse Osmosis membranes made from semi permeable material such as:
Cellulose tri Acetate
Composite polyamide

Membrane films using;
Pierced membranes
Spiral wound membranes

Commercial examples of semi-permeable films include:
Hytrel®
Surlyn®
Crastin®
Imron®
CA (cellulose acetate)

The at least one absorbent pad in the transdermal patch may include materials including, but is not limited to, the following list of materials:

| | |
|---|---|
| Cellulose Fiber Pad | Cotton |
| Natural Sponge | Woven Cloth Fabrics |
| Polyurethane foams | Polyisocynurate Foams |
| Non-Woven Cloths | Fumed Silica |
| Starch | Corn Meal |
| Wood Pulp fibers | Collagen Pads |
| Poly methyl methacrylate | Polyvinyl alcohol |
| Poly vinyl pyrrolidine | Poly acrylic acid |
| Poly (2-hydroxy ethyl methacrylate | Polyacrylamide |
| Poly ethylene glycol | Polylactides(PLA) |
| Polyglycolides(PGA) | Nylon |
| Poly(lactide-Co-glycolides) | Polypropolene |
| Polycarbonate | Chitosan |
| Poly (N-isopropylacrylamide) | |

Co-Polymer formulations of Poly methacrylic acid and Poly ethylene glycol Co-Polymer formulations of Poly acrylic acid and Poly (N-isopropylacrylamide) Hyrdogels, e.g. Polyacrylamide, poly(propylene oxide Pluronic polyols family of gel materials, e.g. Pluronic-chitosan hydrogels Silica gels

It is to be understood that the at least on pad could also be made of any other natural or synthetic material, which will act to absorb the drug compound and be able to release the drug upon ultrasonic excitation.

In some embodiments, the use of an absorbent pad is made to provide extended delivery of the substance via the manipulation of the thickness of the absorbent material, or through the selection of materials with increased absorbency power, thereby enabling the absorbent pad to hold and reserve greater quantities or doses of the substance to be delivered, for a longer period of time.

In some embodiments of the invention, the delivery rate of a substance from the transdermal patch can be adjusted due to the use of an absorbent pad via the manipulation of the thickness of the absorbent material, or through the selection of materials with increased or decreased absorbency power, thereby enabling the absorbent pad to liberate the substance at differing delivery rates form the patch.

In some embodiments of the transdermal patch the use of an absorbent pad provides enhanced resistance to incidental contact between the stored substance and other materials or compounds within the patch construction which could contaminate or degrade the substance, including adhesives used in the fabrication of the patch or to adhere the patch to the patients skin surface.

In some embodiments, the invention further includes, a means of providing regulated and controlled doses of insulin and other medications for the treatment of diabetes, involving a wearable ultrasonic transmitter which is connected to a transdermal patch wherein the patch has been loaded with insulin or other medication for the treatment of diabetes. The combination device acts to regulate the dose delivered to a diabetic patient for the purpose of reducing and controlling serum glucose levels in the diabetic patient.

In some embodiments, the invention includes a combination system that includes a wearable ultrasonic transmitter which is connected to a transdermal patch for the purpose of providing regulated and controlled doses of insulin and other medications for the treatment of diabetes, wherein the insulin loaded patch is used either in conjunction with or in replacement of oral diabetic medication, for night time use, daytime use or both, for the purpose of reducing and controlling serum glucose levels in a diabetic patient.

The invention further includes an enhanced ultrasonic drug delivery transdermal patch suitable for ultrasonic drug delivery, containing an absorbent compound as a means for storing a substance, including but not limited to medication, drugs or nutrient compounds within the patch, wherein the absorbent compound is made to be more resonance compatible with the frequency and intensity of the ultrasonic transmission by pre-treating the absorbent compound to improve its sonic attenuation properties by reducing the quantity of air or gas trapped within the absorbent by: Freezing the absorbent material, and Vacuum drying the absorbent material and/or by Pre-treating the material with sonic energy to remove any impurities within the absorbent material, prior to the application of the substance to the material.

The invention further includes embodiments of a means of instilling a sonic memory into materials used as the semi-permeable film layer of a transdermal patch, wherein the materials are subjected to ultrasound at the desired reactant frequency and intensity levels, while being formulated and cast into a film or membrane state, for a period of time as to make that film or membrane activate its reverse osmosis properties or pore dilation in response to a ultrasonic signal of the same amplitude, frequency and intensity level used during the formulation process.

The invention further includes a modified transdermal delivery device which incorporates a mesh screen at the bottom of the transdermal delivery device, which contacts to the skin, for the purpose of avoiding drug pooling, improving drug absorption, and the speed of absorption of the drug.

The invention further includes a flexible transdermal patch delivery device which incorporates a mesh screen at the bottom of the transdermal delivery device, which contacts the skin, for the purpose of avoiding drug pooling, improving drug absorption, and increasing the speed of absorption of the drug.

The invention further includes embodiments of a transdermal delivery cap or patch-cap delivery device which incorporates a mesh screen at the bottom of the device, which contacts the skin, for the purpose of avoiding drug pooling, improving drug absorption, and increasing the speed of absorption of the drug.

The invention further includes a drug delivery device employing a modified transdermal patch, wherein the modified transdermal patch includes one or more absorbent pads for absorbing a substance, wherein the absorbent material is suitable as a means for storing a substance, including but not limited to a medication, drug or nutrient compound within the patch,

The invention further includes a drug delivery device employing a modified transdermal patch, wherein the modified transdermal patch includes at least two absorbent pads which are stacked upon each other. This embodiment can provide for increased absorption of a substance enabling the patch to (1) hold a greater quantity of the drug, (2) extend the useful life of the patch, (3) Enhance the quantity of the dose which can be released from the patch, by either passive of active methods of drug release.

The invention further includes embodiments of a drug delivery device employing a modified transdermal patch, wherein the modified transdermal patch includes at least one absorbent pad which has a greater thickness to the absorbent material, providing for increased absorption of a substance and enabling the patch to (1) hold a greater quantity of the drug, (2) extend the useful life of the patch, (3) enhance the quantity of the dose which can be released from the patch, by either passive of active methods of drug release.

The absorbent pads in the embodiments of the invention may include material including, but are not limited to, the following materials:

| | |
|---|---|
| Cellulose Fiber Pad | Cotton |
| Natural Sponge | Woven Cloth Fabrics |
| Polyurethane foams | Polyisocynurate Foams |
| Non-Woven Cloths | Fumed Silica |
| Starch | Corn Meal |
| Wood Pulp fibers | Collagen Pads |
| Poly methyl methacrylate | Polyvinyl alcohol |
| Poly vinyl pyrrolidine | Poly acrylic acid |
| Poly (2-hydroxy ethyl methacrylate | Polyacrylamide |
| Poly ethylene glycol | Polylactides(PLA) |
| Polyglycolides(PGA) | Nylon |
| Poly(lactide-Co-glycolides) | Polypropolene |
| Polycarbonate | Chitosan |
| Poly (N-isopropylacrylamide) | |

Co-Polymer formulations of Poly methacrylic acid and Poly ethylene glycol Co-Polymer formulations of Poly acrylic acid and Poly (N-isopropylacrylamide) Hyrdogels, e.g. Polyacrylamide, poly(propylene oxide Pluronic polyols family of gel materials, e.g. Pluronic-chitosan hydrogels Silica gels

In addition the pads may include any other natural or synthetic material, which will act to absorb the drug, compound and be able to release the drug upon ultrasonic excitation.

Having described the invention in the above detail, those skilled in the art will recognize that there are a number of variations to the design and functionality for the device, but such variations of the design and functionality are intended to fall within the present disclosure.

1-24. (canceled)

25. A method for transporting a drug through skin of an individual, comprising applying ultrasound through a transdermal delivery device which is affixed to a programmable ultrasonic regulator device, which itself is worn by the individual wherein the ultrasound is applied at a frequency and intensity and for a time period effective to enable movement of a therapeutic quantity of the drug from a transdermal delivery device, or transdermal patch, through the skin, for the purpose of effecting regulated, and timed drug delivery to the individual, wherein the wearable, portable sonic device is controllable through programmable automatic or manual operation settings comprising quantity of drug released by the device, time interval of active ultrasonic drug delivery, time interval between ultrasonic drug deliveries, frequency and intensity of the ultrasound, a basal delivery schedule of drug dosing and a bolus delivery schedule of booster doses of drug delivery.

26. The method of claim 1, wherein the ultrasound has a frequency in the range of about 20 kHz to 10 MHz. and an intensity in the range of about 0.01 W/cm² to 5.0 W/cm, wherein the ultrasound is applied either in a continuous pulsed manner.

27. The method of claim 1, wherein the drug is insulin and the basal delivery schedule of insulin to the subject for the treatment of diabetes.

28. A method of instilling a sonic memory into materials used as a semipermeable film layer or membrane of a transdermal patch, wherein the materials are subjected to ultrasound at a reactant frequency and intensity level, while being formulated and cast into a film or membrane state, for a period of time as to make the film or membrane activate its reverse osmosis properties or pore dilation in response to an ultrasonic signal of the same amplitude, frequency and intensity level used during the formulation process.

29. A method of enhanced drug delivery employing a modified transdermal patch, wherein the modified transdermal patch comprises at least two absorbent pads or layers which are stacked upon each other, providing for increased absorption of a substance enabling the patch to (1) hold a greater quantity of the drug, (2) extend the useful life of the patch, (3) enhance the quantity of the dose which can be released from the patch, by either passive or active methods of drug release.

30. The method of claim 5, wherein the at least two stacked absorbent pads or layers are stacked without adhesive being in a path of drug delivery through the patch to avoid interaction between a drug delivered through the patch and adhesive.

31. The method of claim 5, wherein the at least two absorbent pads or layers are at least partially separatable such that an airspace can be created between at least a portion of the at least two pads or layers.
